(19) United States
(12) Patent Application Publication
(10) Pub. No.: US 2023/0133956 A1
(43) Pub. Date: May 4, 2023

(54) BONE CONDUCTION BODY SUPPORT DEVICE

(71) Applicant: Philip Root, Boca Raton, FL (US)

(72) Inventor: Philip Root, Boca Raton, FL (US)

(21) Appl. No.: 17/516,144

(22) Filed: Nov. 1, 2021

(51) Int. Cl. H04R 1/02; A61M 21/02; A47G 9/10; G06F 3/16

(52) U.S. Cl. CPC H04R 1/028; A61M 21/02; A47G 9/10; G06F 3/165; A61M 2021/0027

(57) ABSTRACT

A bone conduction body support device is disclosed, including a body support device including a controller releasably engaged to a sidewall of the body support device. A first bone conductor is flexibly engaged to the body support device via a first string connected to a first eyelet positioned in an interior cavity of the body support device. A second bone conductor is flexibly engaged to the body support device via a second string connected to a second eyelet positioned in the interior cavity of the body support device. Each of the first bone conductor and the second bone conductor connected to the controller via an electrical wiring.

### CROSS-REFERENCE TO RELATED APPLICATIONS

This application references U.S. Pat. No. 10,751,503 B2 entitled “BONE CONDUCTION BODY SUPPORT SYSTEM”, the entire disclosure of which is incorporated herein by reference in its entirety.

### TECHNICAL FIELD

Embodiments of the invention relate to devices for providing vestibular and somatosensory stimulation utilizing bone conduction technologies, and more specifically relates to attachment mechanisms to flexibly engage a bone conductor with a pillow.

### BACKGROUND

The human body can sense sound by air conduction as well as bone conduction. While air conduction is how we typically converse and hear our surroundings, bone conduction can also stimulate the vestibular and somatosensory systems as sound vibration is transmitted through the body's skeletal framework.

Stimulating these sensory systems using bone conduction has been proven to result in a variety of benefits including the promotion of neurological development, relaxation and stabilization of the body. Auditory devices have also been used to help treat and/or prevent ADHD, autism, Asperger's, and sensory processing disorder among others. These auditory devices have consisted of air conduction headphones as well as bone conduction devices attached to the user's headphones. While these have been effective, these devices may be uncomfortable. Further, it has been shown that vestibular stimulation in-utero provides similar benefits to the fetus, for which prior art devices are not practical.

### SUMMARY OF THE INVENTION

This summary is provided to introduce a variety of concepts in a simplified form that is disclosed further in the detailed description of the embodiments. This summary is not intended to identify key or essential inventive concepts of the claimed subject matter, nor is it intended for determining the scope of the claimed subject matter.

Embodiments described herein provide for a bone conduction body support device, including a body support device including a controller releasably engaged to a sidewall of the body support device. A first bone conductor is flexibly engaged to the body support device having a first string connected to a first eyelet positioned in an interior cavity of the body support device. A second bone conductor is flexibly engaged to the body support device via a second string connected to a second eyelet positioned in the interior cavity of the body support device. Each of the first bone conductor and the second bone conductor connected to the controller via an electrical wiring. The flexibly engaged first and second bone conductors and controllers allow the first and second bone conductors and the controller to be removed when cleaning the pillow and/or the cover.

A bone conduction body support is provided having a body support device including a controller releasably engaged to a strap encircling the body support device. A first bone conductor and a second bone conductor are each flexibly engaged to the body support device via the strap to allow the first and second bone conductors. The foam batting of the body support device (e.g., a pillow, body pillow, etc.) allows the bone conductors to flex while providing a comfortable surface for the user's head and/or body to rest on. In an embodiment wherein a string is utilized, the foam batting allows the body support device to compress and allows the bone conductors to rotate and/or articulate to conform to the user's head.

The pillow may be filled with a foam batting which provides a contoured shape to the pillow. The foam batting increases the rigidity of the pillow and allows for the controller to be mounted to the sidewall or strap of the body support device.

### BRIEF DESCRIPTION OF THE DRAWINGS

A more complete understanding of the embodiments, and the attendant advantages and features thereof, will be more readily understood by references to the following detailed description when considered in conjunction with the accompanying drawings wherein:

FIG. 1 illustrates a perspective view of the body support device having a flexibly engaged bone conduction assembly, according to an embodiment of the present invention;

FIG. 2 illustrates a perspective view of the body support device having a flexibly engaged bone conduction assembly, according to an embodiment of the present invention;

FIG. 3 illustrates a perspective view of the controller to control the functions of the bone conductor, according to an embodiment of the present invention;

FIG. 4 illustrates a perspective view of the bone conductor, according to an embodiment of the present invention;

FIG. 5 illustrates a perspective view of the body support device having a flexibly engaged bone conduction assembly and the controller releasably engaged with the body support device, according to an embodiment of the present invention;

FIG. 6 illustrates a perspective view of the body support device having a flexibly engaged bone conduction assembly, according to an embodiment of the present invention;

FIG. 7 illustrates a cutaway view of the bone conductors and attachment mechanism thereof, according to an embodiment of the present invention; and

FIG. 8 illustrates a side elevation view of the body support device having a flexibly engaged bone conduction assembly, according to some embodiments.

### DETAILED DESCRIPTION

The specific details of the single embodiment or variety of embodiments described herein are set forth in this application. Any specific details of the embodiments are used for demonstration purposes only, and no unnecessary limitation or inferences are to be understood therefrom.

Before describing in detail exemplary embodiments, it is noted that the embodiments reside primarily in combinations of components related to the system. Accordingly, the device components have been represented where appropriate by conventional symbols in the drawings, showing only those specific details that are pertinent to understanding the embodiments of the present disclosure so as not to obscure the disclosure with details that will be readily apparent to those of ordinary skill in the art having the benefit of the description herein.

The present embodiments provide for a body support structure having integrated bone conductors and associated electrical components in communication thereto. Each bone conductor is comprised of at least one transducer configured to transmit stimulation to the skeletal structure of the user. Each bone conductor is embedded within a bodily support device, and depending on its overall size, may be placed underneath, on top of, or around a user whom is receiving the auditory stimulation. The body support device may be constructed from a few inches square or any larger size. Each transducer is applied to permit the conduction of sound waves to any part of the user's skeletal framework. The bodily support device having embedded transducers may also be suitably applied to conduct sound to a fetus (secondary user).

Each bone conductor is at least partially embedded into the foam batting of the body support device. In such, the cover of the body support device (e.g., a pillowcase or pillow cover) can be removed to allow the user to clean or replace the cover. The bone conductors will then remain in a suitable position on the body support device without needing readjustment.

Each bone conductor is flexible on the surface of the body support device while the conductors are isolated from vibrating the attachment components (e.g., the eyelets, string, etc.). For example, if a user touches a bone conductor with rigid plastic or hard glue, the substrate will vibrate, causing the sound to bleed. While other methods of attachment are possible, keeping the bone conductor's flexible allows them to be quiet and compressible into the foam and remain in the correct orientation. Further, the flexibly mounted bone conductors allow for positive contact to be made with about 3-7 pounds of pressure while remaining comfortable for the user.

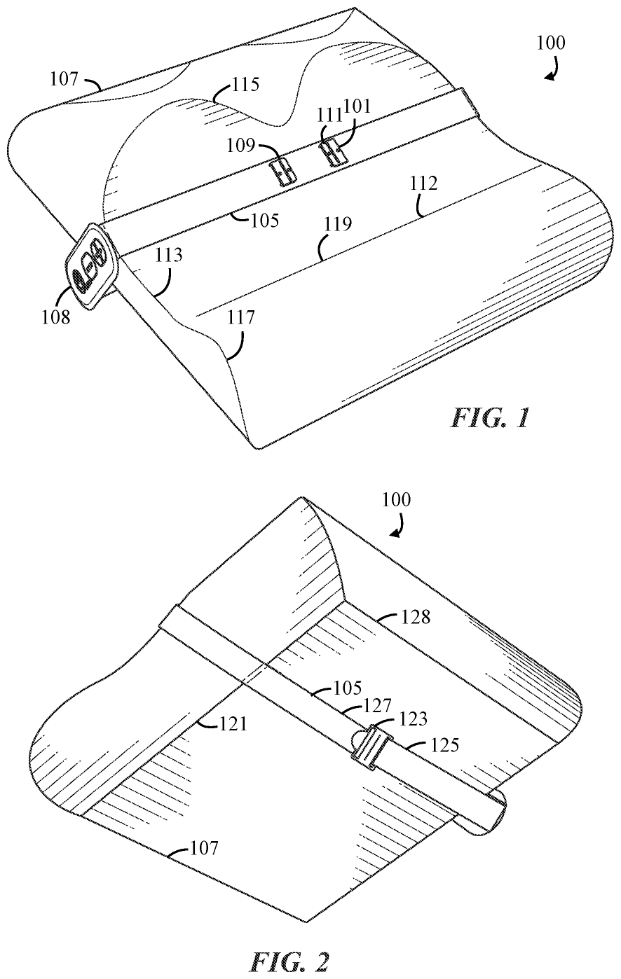

FIG. 1 illustrates a perspective view of the body support device 100 having a flexibly engaged bone conduction assembly 101 and controller 108 electrically connected thereto. The bone conduction assembly 101 includes a strap 105 which encircles the body support device 100. In the present embodiment, the body support device is illustrated as a pillow 107. The strap 105 is constructed of a flexible material to encircle the body support device 100 and allow the user to remove the strap 105 when washing the body support device 100. The strap 105 is releasably engaged with a controller 103 positioned along the length of the strap 105. The controller 108 may be releasably engaged via a quick release mechanism, snap fit mechanism, or other means of releasable attachment. The strap 105 includes a first conductor attachment 109 and a second conductor attachment 111 each to flexibly engage with a bone conductor. In some embodiments, the bone conductors are attached to a top 112 of the strap 105.

In some embodiments, the pillow 107 is a contour pillow having an ergonomically contoured portion 113 recessed from a top portion 115 and a bottom portion 117. The contoured portion 109 receives the head of the user and provides added comfort. The contoured portion 113 is positioned on the top side 119 of the pillow 107.

FIG. 2 illustrates a perspective view of the bottom side 121 body support device 100 having a flexibly engaged bone conduction assembly 101. The bone conduction assembly 101 is releasably engaged via a clasp 123 which releasably attaches a first end 125 of the strap 105 with a second end 127 of the strap 105. During use, such as when the user is cleaning the pillow cover 128 or pillow 107, the clasp 123 is disengaged and removed.

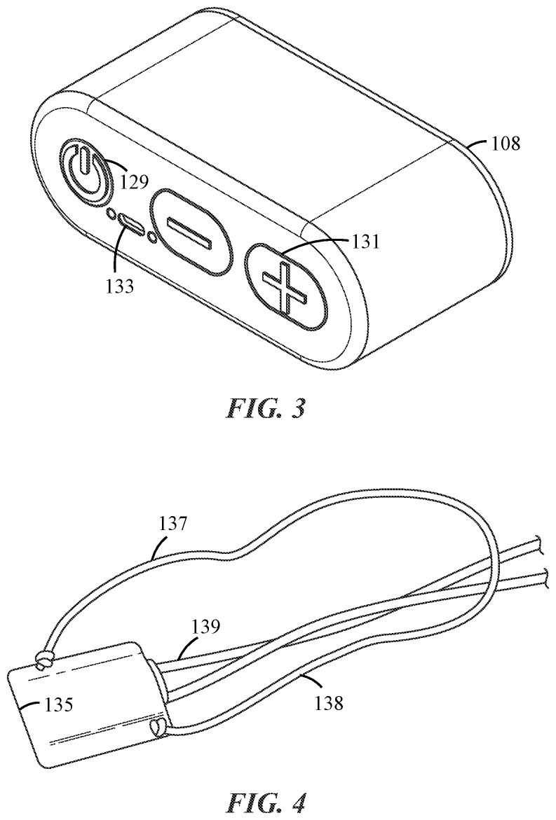

FIG. 3 illustrates a perspective view of the controller 108 to control the functions of the bone conductor. The controller 108 is in electrical communication with the bone conductor. The controller 108 is comprised of a ON/OFF button 129, volume controls 131, a battery indicator and a signal indicator as well as other controls known in the arts. Further, the controller 108 may include a power input port 133 to receive a charging cable, universal serial bus (USB) port, etc.

FIG. 4 illustrates a perspective view of the first bone conductor 135 and the second bone conductor 136 attached to a first string 137 and a second string 138 which each to the first conductor attachment 109 (see FIG. 1) and second conductor attachment 111 (see FIG. 1) respectively. The strings 137,138 allows for each bone conductor 135,136 to be retained in position on the pillow 107 when the cover is washed or otherwise cleaned. Electrical wiring 139 extends from each bone conductor 135,136 to the controller 108 (see FIG. 3). The electrical wiring 139 may provide electrical power to each bone conductor 135,136, transmit an electrical signal to the controller 108, and/or receive electrical signals from the controller 108.

In some embodiments, the strings 137,138 to the bottom side 121 of the foam batting works well because when compressed, the foam batting pushes the conductors 135,136 against the head and lets the conductor rotate if needed to confirm the user's head. Then when the user removes their head from the conductor, it is forced back to the surface of the foam.

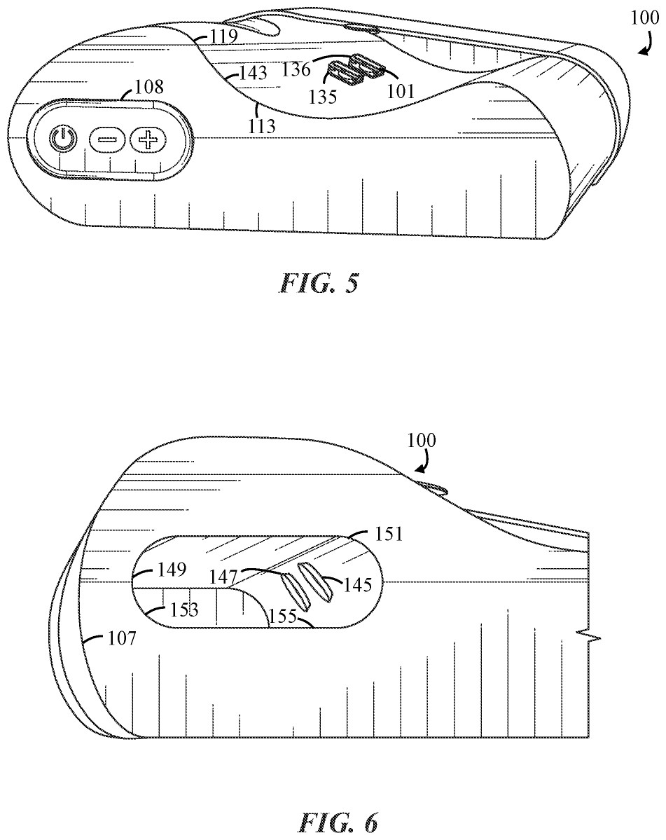

FIG. 5 illustrates a perspective view of the body support device 100 having a flexibly engaged bone conduction assembly 101 and the controller 108 releasably engaged with the body support device 100. In the embodiment illustrated in FIG. 5, the controller 108 is at least partially embedded into a sidewall 141 of the body support device 100. The controller 108 may be retained at least partially within a cavity to allow the user to remove the controller 108 when washing the body support device 100. Each bone conductor 135,136 is positioned on the upper surface 143 of the top side 119. Each bone conductor 135,136 is positioned at the contoured portion 113 such that each bone conductor 135,136 is removed from contact with the cover of the pillow 107 when the users head is not resting thereon.

In some embodiments, the controller may be permanently affixed, such as via glue, anchor, or other attachment means.

FIG. 6 and FIG. 8 illustrate the body support device 100 wherein each bone conductor 135,136 is flexibly attached to the body support device and each connected to a first eyelet 145 and a second eyelet 147 at least partially embedded within the oblong portion 149 of the pillow 107. FIG. 6 illustrates the oblong portion 149 having been removed from the pillow 107 to illustrates the first eyelet 145 and second eyelet 147 positioned on the top 151 of the interior 153 of the cavity 155 formed once the oblong portion 149 is removed. In such, each bone conductor 135,136 is attached to the first eyelet 145 and the second eyelet 147 via the strings 137,138 which extends through the batting of the pillow 107. As shown in FIG. 5, the controller 108 may be positioned at least partially within the cavity 155 (see FIG. 6) within the oblong portion 149.

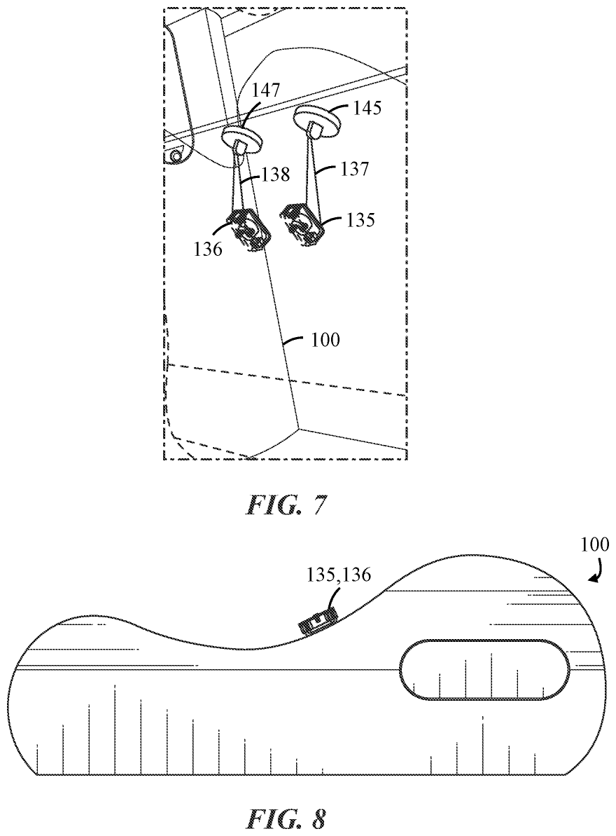

FIG. 7 illustrates a cutaway view of each bone conductor 135,136 connected to the first eyelet 145 and the second eyelet 147 via the strings 137,138. The strings 137,138 extend through the batting of the body support device 100 and allows for the removal of the cover without moving the bone conductors 135,136.

Suitable application positions of the bone conductors include bodily regions such as the cranium, spine, hip, or leg bones. One skilled in the arts may appreciate that any point along the skeletal framework of the user may be effective in conducting sound waves to the user's vestibular and/or somatosensory systems. To permit modulation of the position of the attachment mechanism (e.g., the first eyelet 145, second eyelet 147, strap 105, or other attachment mechanism) can be moved at any point on the body support device 100. In some embodiments a mark on the surface of the removable cover can indicate an intended target for the transducers contact point. This mark may call out body parts such as “HEAD” to indicate correct placement.

In some embodiments, the one or more bone conductors are utilized to provide sound wave bone conduction in frequencies primarily between 50 and 4,000 Hertz (“Hz”).

In another embodiment, each bone conductor 135,136 may be made in differing sizes and thicknesses to permit each bone conductor 135,136 to be used in a variety of body support device 100 configurations not limited to pillows. Alternative embodiments may include bedding, massage tables and chairs as well as other commercially and privately used support devices.

The embodiments illustrate a pair of bone conductors which may be positioned to engage any points on the user's body where the vestibular/somatosensory systems may be stimulated. It may be preferential for each bone conductor 135,136 to be placed on each temple of the user.

In order for sound to be transmitted through each bone conductors 135,136, a recording playback device may be in communication with the controller 108. Sound sources may include CD players, MP3 players, tape players, RF transmitter connected to a device such as a TV, or other sources of sound well known in the arts. Further, a Near Frequency Communication (NFC) capable chip may be embedded within the device may permit any device having wireless connectivity capabilities to be utilized including portable and static computer systems, PDA's, tablets, and mobile phones to be utilized in the transmission of sound to the device.

In some embodiments, the attachment mechanism may include a spike which allows the bone conductors to flex, while the point creates additional pressure on the bone conductors. This helps to create additional pressure when soft foams are used for the batting.

In some embodiments, soft hot glue or flexible spray adhesive allows for the same flexible mounting of the bone conductors and will aid in creating additional pressure when using soft foam batting.

For example, an amplifier operates in response to user input that controls the application of music tonal frequencies to the amplifier in communication with the bone conductors 135,136. This can be achieved by using a hard-wired control, or a wireless control. The wireless control can use RF signals, IR signals, etc. Control supplies the source of music and controls the application of the source of music to the amplifier and bone conductors 135,136.

Sounds selected for transmission may include any embodiment typically utilized in the arts including full-spectrum music, low frequency sound, predetermined frequency filtered sounds, recordings of physiological and anatomical sounds among other rhythmic or otherwise therapeutic sounds having the desired neurological, physiological, or other stimulatory effects.

In an embodiment, the filtered and treated audio data are loaded onto a recording playback device, such as an iPod™ device or similar electronic data storage implement. In this embodiment, a wireless connection between the treated audio data stored on the recording playback device and a plurality of bone conduction devices permit a group of individuals to share the same bone conduction signal in a group setting, or likewise in a solitary setting.

One skilled in the art may appreciate that both mono and multiple channel audio configurations may be utilized depending on the type of audio broadcast. A stereo or multiple channel configuration may be preferable if a plurality of bone conduction transducers are used as well as the intended effect, weather for auditory pleasure or therapy.

Another type of bone conductor that can be used to transmit music and tones to the surface of the body is an electro-active polymers (EAPs). EAPs are disclosed in an article entitled “Artificial Muscles” by Steven Ashley, Scientfic American, October 2003, pp. 53-59. Electro-active polymers are polymers that move in response to an electrical current. As disclosed in the Scientific American article, supra,
“The fundamental mechanism underlying new artificial muscle products is relatively simple. When exposed to high-voltage electric fields, dielectric elastomers—such as silicones and acrylics—contract in the direction of the electric field lines and expand perpendicularly to them, a phenomenon physicists term Maxwell stress.

The exterior surface of the body support device may be constructed of any fabric known in the textile arts commonly associated with pillows. This may include cotton, polyester, silk, or blends of any commonly utilized materials.

The batting 157 may be foam, memory foam, foam beads, foam particles, etc. The batting 157 being made of a foam material allows the controller 108 to be rigidly mounted to the body support device 100.

Many different embodiments have been disclosed herein, in connection with the above description and the drawings. It will be understood that it would be unduly repetitious and obfuscating to literally describe and illustrate every combination and subcombination of these embodiments. Accordingly, all embodiments can be combined in any way and/or combination, and the present specification, including the drawings, shall be construed to constitute a complete written description of all combinations and subcombinations of the embodiments described herein, and of the manner and process of making and using them, and shall support claims to any such combination or subcombination.

It will be appreciated by persons skilled in the art that the present embodiment is not limited to what has been particularly shown and described hereinabove. A variety of modifications and variations are possible in light of the above teachings without departing from the following claims.

What is claimed is:

1. A bone conduction body support device, comprising:
a body support device including a controller releasably engaged to a sidewall of the body support device;
a first bone conductor flexibly engaged to the body support device via a first string connected to a first eyelet positioned in an interior cavity of the body support device; and
a second bone conductor flexibly engaged to the body support device via a second string connected to a second eyelet positioned in the interior cavity of the body support device, each of the first bone conductor and the second bone conductor connected to the controller via an electrical wiring.

2. The bone conduction body support device of claim 1, wherein the body support device is a pillow.

3. The bone conduction body support device of claim 2, wherein the pillow includes a contoured portion.

4. The bone conduction body support device of claim 3, wherein the interior cavity is positioned in an oblong portion of the pillow.

5. The bone conduction body support device of claim 4, wherein the first bone conductor and the second bone conductor are positioned on a top side of the pillow.

6. The bone conduction body support device of claim 5, wherein the first bone conductor and the second bone conductor are each positioned at the contoured portion such that each the first bone conductor and the second bone conductor are selectively removed from contact with a pillow cover.

7. The bone conduction body support device of claim 6, wherein the controller includes an ON/OFF button.

8. The bone conduction body support device of claim 7, wherein the controller includes one or more volume controls.

9. The bone conduction body support device of claim 8, wherein the first string and the second string extend through a batting of the pillow.

10. The bone conduction body support device of claim 9, wherein the batting is a foam.

11. A bone conduction body support device, comprising:
a body support device including a controller releasably engaged to a strap encircling the body support device;
a first bone conductor and a second bone conductor each flexibly engaged to the body support device via the strap, each of the first bone conductor and the second bone conductor connected to the controller via an electrical wiring.

12. The bone conduction body support device of claim 11, wherein the strap is releasably engaged with the body support device via a clasp.

13. The bone conduction body support device of claim 12, wherein the body support device is a pillow.

14. The bone conduction body support device of claim 13, wherein the pillow includes a contoured portion.

15. The bone conduction body support device of claim 14, wherein the first bone conductor and the second bone conductor are each positioned on a top of the strap.

16. The bone conduction body support device of claim 15, wherein the controller includes an ON/OFF button.

17. The bone conduction body support device of claim 16, wherein the controller includes one or more volume controls.

18. The bone conduction body support device of claim 17, wherein the controller includes a power input port.

19. The bone conduction body support device of claim 18, wherein the pillow includes a foam batting.

20. The bone conduction body support device of claim 19, wherein the clasp is positioned at the bottom side, and wherein the clasp connects a first end of the strap to a second end of the strap.
